(19) United States
(12) Patent Application Publication
(10) Pub. No.: US 2026/0148866 A1
(43) Pub. Date: May 28, 2026

(54) MACHINE LEARNING SYSTEMS AND RELATED ASPECTS FOR CLASSIFYING INDETERMINATE BILIARY STRICTURES

(71) Applicant: THE JOHNS HOPKINS UNIVERSITY, Baltimore, MD (US)

(72) Inventors: Mouen A. KHASHAB, Baltimore, MD (US); Gregory D. HAGER, Baltimore, MD (US); Venkata S. AKSHINTALA, Towson, MD (US); Satyanarayana S. VEDULA, Baltimore, MD (US)

(21) Appl. No.: 19/122,851

(22) PCT Filed: Oct. 23, 2023
(86) PCT No.: PCT/US2023/035691

§ 371 Date: Apr. 21, 2025

Related U.S. Application Data
(60) Provisional application No. 63/380,727, filed on Oct. 24, 2022.

(51) Int. Cl. G16H 70/60

(52) U.S. Cl. CPC G16H 70/60

(57) ABSTRACT

Examples may provide an electronic neural network that has been trained on a set of training data that comprises a plurality of sets of features extracted from image data of biliary strictures and clinical data obtained from reference subjects. The electronic neural network is configured to output a positive or negative disease state classification of the biliary stricture in a test subject that is indicated by a set of features extracted from image data of biliary strictures and clinical data obtained from the test subject when the set of features is passed through the electronic neural network. Related computer readable media and methods are also provided.

### CROSS-REFERENCE TO RELATED APPLICATIONS

This application is the national stage entry of International Patent Application No. PCT/US2023/035691, filed on Oct. 23, 2023, and published as WO 2024/091441 A1 on May 2, 2024, which claims the benefit of U.S. Provisional Patent Application Ser. No. 63/380,727, filed on Oct. 24, 2022, which are hereby incorporated by reference herein in their entireties.

### FIELD

This disclosure relates generally to machine learning, e.g., in the context of medical applications, such as pathology.

### BACKGROUND

Bile duct cancer or cholangiocarcinoma typically presents as biliary strictures. These strictures remain a diagnostic dilemma since a significant proportion of them remain indeterminate for malignancy despite radiologic, endoscopic, and laboratory testing. Indeterminate biliary strictures have challenging course with more than 25% of patients who are presumed to have malignant strictures, after undergoing major surgery end up having benign pathology. Recently developed spyglass cholangioscopy has provided direct visualization and histological examination of bile duct but accuracy in differentiating benign and malignant strictures continues to be poor.

Accordingly, it is apparent that there is a need for additional methods of classifying disease states of biliary strictures in test subjects.

### SUMMARY

The present disclosure provides, in certain aspects, an artificial intelligence (AI) system capable of classifying a disease state of indeterminate biliary strictures in test subjects. In some aspects, for example, the present disclosure provides a computational framework for classifying a disease state (e.g., malignant or benign) of an indeterminate biliary stricture in test subject that uses electronic neural networks that have been trained with features extracted from image data of indeterminate biliary strictures and clinical data obtained from reference subjects. These and other aspects will be apparent upon a complete review of the present disclosure, including the accompanying figures.

According to various embodiments, a computer-implemented method of classifying a disease state of a biliary stricture in a test subject is presented. The method includes: passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and outputting from the electronic neural network a positive or negative disease state classification of the biliary stricture in the test subject indicated by the set of features extracted from the image data of the biliary stricture and the clinical data obtained from the test subject.

Various optional features of the above embodiments include the following. An accuracy level of the positive or the negative disease state classification output from the electronic neural network is higher than an accuracy level of the positive or the negative disease state classification output from another electronic neural network trained only on the plurality of sets of features extracted from image data of biliary strictures. Generating a therapy recommendation for the test subject based upon the positive or negative disease state classification of the biliary stricture in the test subject output from the electronic neural network. Administering, or discontinuing administering, a therapy to the test subject based upon the positive or negative disease state classification of the biliary stricture in the test subject output from the electronic neural network. The positive disease state comprises a cancerous disease state and the negative disease state comprises a benign disease state. The clinical data comprises demographic data, symptom data, physical examination data, or a combination thereof. The clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level. The image data comprises cholangioscopy images and/or videos of the biliary strictures obtained from the test subject and the reference subjects using a cholangioscope. The biliary strictures of the reference subjects comprise indeterminate biliary structures. The features extracted from the image data comprise indicators of papillary mass, dilated vessels, tortuous vessels, ulceration, or a combination thereof. The features comprise numerical vectors. The passing and outputting steps are performed substantially in real-time with obtaining the image data of the biliary stricture from the test subject.

According to various embodiments, a system for classifying a disease state of a biliary stricture in a test subject using an electronic neural network is presented. The system includes a processor; and a memory communicatively coupled to the processor, the memory storing non-transitory computer-executable instructions which, when executed on the processor, perform operations including: passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and outputting from the electronic neural network a positive or negative disease state classification of the biliary stricture in the test subject indicated by the set of features extracted from the image data of the biliary stricture and the clinical data obtained from the test subject.

Various optional features of the above embodiments include the following. The instructions which, when executed on the processor, further perform operations comprising: generating a therapy recommendation for the test subject based upon the positive or negative disease state classification of the biliary stricture in the test subject output from the electronic neural network. An accuracy level of the positive or the negative disease state classification output from the electronic neural network is higher than an accuracy level of the positive or the negative disease state classification output from another electronic neural network trained only on the plurality of sets of features extracted from image data of biliary strictures. The positive disease state comprises a cancerous disease state and wherein the negative disease state comprises a benign disease state. The clinical data comprises demographic data, symptom data, physical examination data, or a combination thereof. The clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level. The image data comprises cholangioscopy images and/or videos of the biliary strictures obtained from the test subject and the reference subjects using a cholangioscope. The biliary strictures of the reference subjects comprise indeterminate biliary structures. The features extracted from the image data comprise indicators of papillary mass, dilated vessels, tortuous vessels, ulceration, or a combination thereof. The features comprise numerical vectors.

According to various embodiments, a computer readable media is presented. The computer readable media comprises non-transitory computer-executable instructions which, when executed by at least one electronic processor, perform at least: passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and outputting from the electronic neural network a positive or negative disease state classification of the biliary stricture in the test subject indicated by the set of features extracted from the image data of the biliary stricture and the clinical data obtained from the test subject.

Various optional features of the above embodiments include the following. The instructions which, when executed by the electronic processor, further perform operations comprising: generating a therapy recommendation for the test subject based upon the positive or negative disease state classification of the biliary stricture in the test subject output from the electronic neural network. The positive disease state comprises a cancerous disease state and wherein the negative disease state comprises a benign disease state. The clinical data comprises demographic data, symptom data, physical examination data, or a combination thereof. The clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level. The image data comprises cholangioscopy images and/or videos of the biliary strictures obtained from the test subject and the reference subjects using a cholangioscope. The biliary strictures of the reference subjects comprise indeterminate biliary structures. The features extracted from the image data comprise indicators of papillary mass, dilated vessels, tortuous vessels, ulceration, or a combination thereof. The features comprise numerical vectors.

### DRAWINGS

The above and/or other aspects and advantages will become more apparent and more readily appreciated from the following detailed description of examples, taken in conjunction with the accompanying drawings, in which:

FIG. 1 is a flow chart that schematically shows exemplary method steps of classifying a disease state of a biliary stricture in a test subject according to some aspects disclosed herein;

FIG. 2 is a schematic diagram of an exemplary system suitable for use with certain aspects disclosed herein;

FIG. 3 schematically depicts aspects of a machine learning process for classification of indeterminate biliary strictures using cholangioscopy images and a patient clinical data model according to an exemplary embodiment; and,

FIG. 4 schematically shows a study flow chart of patient allocation according to an exemplary embodiment.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Biliary Stricture: As used herein, “biliary stricture” or “bile duct stricture” refers a narrowed segment of the extrahepatic or intrahepatic biliary ductal system. A biliary stricture can impede the normal antegrade flow of bile, which may cause proximal dilatation resulting in clinical and pathological sequelae of biliary obstruction.

Classifier. As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., medical images or the like).

Labeled: As used herein, “labeled” in the context of data sets or points refers to data that is classified as, or otherwise associated with, having or lacking a given characteristic or property.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as a malignancy and/or the like).

Value: As used herein, “value” generally refers to an entry in a dataset that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. INTRODUCTION

In some aspects, the present disclosure provides machine learning based software tools that combine patient information and cholangioscopy images/videos to classify biliary strictures as benign or malignant. The true incidence of benign or malignant biliary strictures is unknown. However, the common etiologies of malignant biliary strictures, such as cholangiocarcinoma (bile duct stricture is 1.6 per 100,000 and for pancreatic cancer is 7.6 per 100,000 in the U.S. Currently available diagnostic techniques to determine the presence of malignancy in biliary strictures has around 75% accuracy. The software or computer readable media and other aspects disclosed herein can improve this accuracy level.

In some embodiments, the machine learning based image processing software of the present disclosure provides guidance to an endoscopist regarding the presence of malignancy within the indeterminate bile duct strictures, which otherwise cannot be easily differentiated by the endoscopist using conventional techniques. This software can be used with the cholangioscope processor (e.g., digital single operator cholangioscope system, a fiber optic cholangioscope system, or the like) for real time assessment.

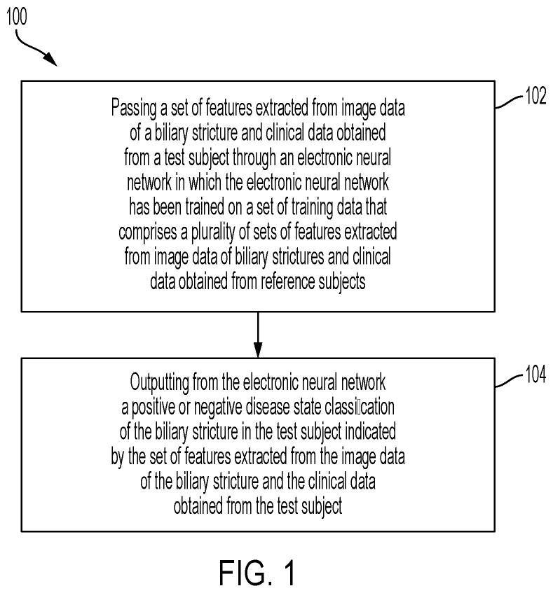

In some aspects, for example, the present disclosure provides computer-implemented methods of classifying a disease state of a biliary stricture in a test subject. To illustrate, FIG. 1 is a flow chart that schematically shows certain of these exemplary method steps. As shown, method 100 includes passing a set of features extracted from image data of the biliary stricture and clinical data obtained from the test subject through an electronic neural network that has been trained on a set of training data that comprises a plurality of sets of features extracted from image data of biliary strictures and clinical data obtained from reference subjects (step 102). Typically, the features extracted from the image data of the biliary stricture and clinical data obtained from the test and reference subjects comprise numerical vectors. The image data of the biliary strictures (e.g., indeterminate biliary structures) and the clinical data obtained from the reference subjects are each labeled with a positive or negative disease state ground truth classification for a given reference subject. Also, one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained. In some embodiments, the image data of the biliary strictures and the clinical data obtained from the test subject is received by a system that comprises the electronic neural network. In some embodiments, the electronic neural network uses one or more algorithms selected from, for example, a random forest algorithm, a support vector machine algorithm, a decision tree algorithm, a linear classifier algorithm, a logistic regression, a linear regression algorithm, a polynomial regression algorithm, or the like. Method 100 also includes outputting from the electronic neural network a positive or negative disease state classification of the biliary stricture in the test subject indicated by the set of features extracted from the image data of the biliary stricture and the clinical data obtained from the test subject (step 104). In some embodiments, method 100 also includes generating a therapy recommendation for the test subject based upon the positive or negative disease state classification of the biliary stricture in the test subject output from the electronic neural network, such as when there is a positive indication of the presence of the disease state in the test subject. In some of these embodiments, method 100 further includes administering a therapy (e.g., a chemotherapy, an immunotherapy, a radiation therapy, or the like) to the test subject based upon the positive or negative disease state classification of the biliary stricture in the test subject output from the electronic neural network. Typically, the positive disease state comprises a cancerous disease state, while the negative disease state comprises a benign disease state. In some embodiments, the passing and outputting steps are performed substantially in real-time with obtaining the image data of the biliary stricture from the test subject.

Various types of image data of biliary strictures and clinical data are optionally utilized in performing the methods of the present disclosure. In some embodiments, for example, the image data comprises cholangioscopy images and/or videos of the biliary strictures obtained from the test subject and the reference subjects using a cholangioscope. In some embodiments, the clinical data comprises demographic data, symptom data, physical examination data, or a combination thereof. In some embodiments, the features extracted from the image data comprise indicators of papillary mass, dilated vessels, tortuous vessels, ulceration, or a combination thereof. Typically, the features comprise numerical vectors.

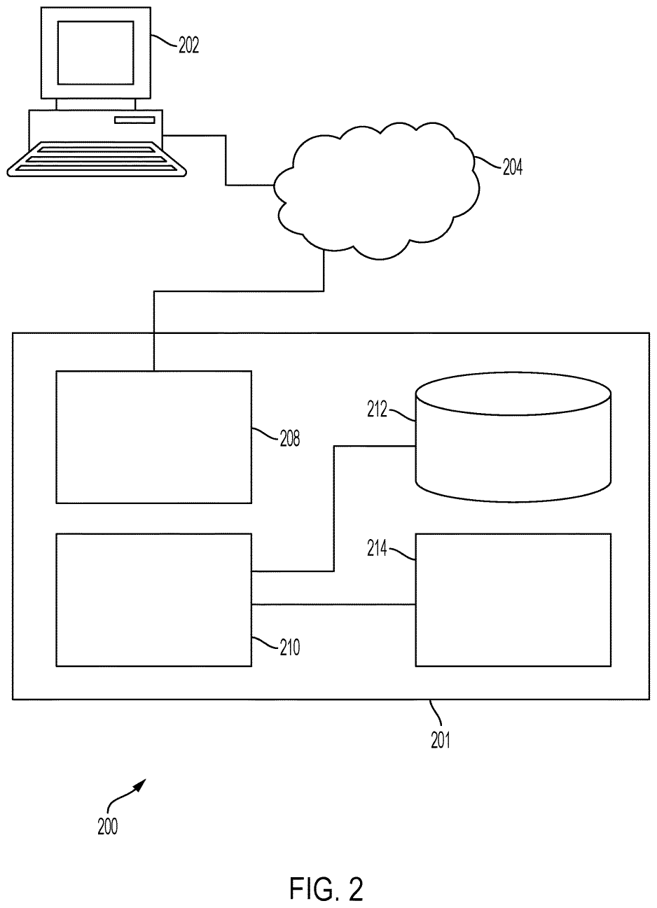

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of image data of biliary strictures and clinical data suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. DESCRIPTION OF EXAMPLE EMBODIMENTS

### Example 1: Machine Learning for Classification of Indeterminate Biliary Strictures During Cholangioscopy

### Introduction

Indeterminate biliary strictures remain a diagnostic challenge despite advancements in radiologic, endoscopic, and laboratory testing. More than 25% of patients presumed to have malignant strictures during cholangioscopy show benign pathology after major surgical intervention. Interpretation of the visual findings during cholangioscopy remains challenging even for experienced endoscopists. We therefore aimed to develop a software tool that classifies indeterminate biliary strictures as benign or malignant using both cholangioscopy images and clinical data.

### Methods

Our dataset included cholangioscopy images and clinical data from a retrospective cohort of patients undergoing cholangioscopy for evaluation of indeterminate biliary strictures. We annotated images for abnormal features suggestive of malignancy, including papillary mass, dilated and tortuous vessels and ulceration. We trained a convolutional neural network (CNN) based on ResNet-18 to detect presence of abnormal image features and tested it in patients of independent centers (external validation). We used multiple outputation to analyze the patient as the unit of analysis and estimated accuracy, sensitivity, specificity, positive and negative predictive values (PPV and NPV), and area under the receiver operating characteristic curve (AUC).

### Results

A total of 1,371,605 cholangioscopy images were obtained from 528 patients at 25 centers (13 North America, 7 Asia, 2 Europe, 2 Australia, 1 South America). Our training set included data from 254 patients at 14 centers, and the test set included data from 95 patients at 8 other independent centers. Table 1 shows the proportion of patients with abnormal cholangioscopy image features according to their final diagnosis. For detection of abnormal image features, the CNN showed a sensitivity of 0.81 (95% confidence interval: 0.72 to 0.91); specificity 0.91 (0.86 to 0.97); PPV 0.93 (0.88 to 0.98); NPV 0.77 (0.66 to 0.88); and AUC 0.86 (0.80 to 0.92).

TABLE 1.1
Proportion of patients with cholangioscopy images
showing abnormal features (suggestive of malignancy)
according to their final diagnosis.

| | |
|---|---|
| Malignant | Benign |
| Biliary Stricture | Biliary Stricture |
| (N = 217) | (N = 132) |

Abnormal Cholangioscopy Image Features

| | | |
|---|---|---|
| Papillary Mass; n (%) | 144 (66.3) | 31 (23.5) |
| Dilated and Tortuous | 79 (36.4) | 10 (7.6) |
| Vessels; n (%) | | |
| Ulceration; n (%) | 51 (23.5) | 10 (7.6) |

### Conclusion

Using data from a large cohort of patients across the world, we trained and externally validated a CNN that can detect key cholangioscopy image features suggestive of malignancy and thus support intra-procedural decision-making. Our next step is to enhance the CNN with clinical data and evaluate it for diagnosing and predicting malignancy in indeterminate biliary strictures. This can improve clinical outcomes through accurate diagnosis of disease and prevention of unwarranted surgical intervention.

### Example 2: Combining Clinical Predictors and Image Features Enhances Cholangioscopy Based Artificial Intelligence Deep Learning for the Diagnosis of Malignant Biliary Strictures

### Introduction

Accurate differentiation of benign from malignant biliary strictures is important for appropriate early management and prevention of unwarranted surgical interventions. However, biliary strictures often remain indeterminate despite various radiologic, laboratory, as well as endoscopic evaluations. Direct visualization of the biliary mucosa can aid in the detection of mucosal abnormalities and allow visually directed tissue sampling of suspicious lesions. The introduction of digital single-operator cholangioscopy has allowed visually guided biopsies and the identification that specific visual findings, such as dilated and tortuous vessels and papillary projections or masses, can be associated with malignancy. Nevertheless, despite high specificities of up to 98% for cholangioscopy-guided biopsies, the pooled sensitivity was as low as 60.1%. Furthermore, although classification systems for DSOC visual findings were proposed, however, they were associated with only fair interobserver agreement (IOA) for presumptive diagnosis based on it.

Deep convolutional neural networks (CNN) are methods of artificial intelligence (AI) that can be used to learn patterns in images that are useful for prediction models. In medicine, the use of AI has been rapidly growing and proving its effectiveness for decision-support in various applications including distinguishing adenomatous from hyperplastic colonic polyps. Digital cholangioscopy provides images of the biliary duct mucosa that can act a source of data for prediction models. Recently, various CNN models have been developed to differentiate malignant from benign biliary strictures during cholangioscopy. The neural networks developed thus far rely solely on analyzing procedural images and videos without incorporating clinical data. Furthermore, these CNN models lack external validity as they are based on data from one or two centers. As such, our objective was to develop a prediction model combining information from cholangioscopy images and clinical data to classify indeterminate biliary strictures, and to test the performance of the model on data from patients at centers that are independent from those used to train it (i.e., external validation or generalizability of the model).

### Methods

Patient selection and study design: We created a prospective cohort of patients at multiple centers across the world who underwent cholangioscopy using Spyglass DS I or II between **/20** and **/20** for evaluation of indeterminate biliary strictures. Patients who underwent cholangioscopy for evaluation or treatment of biliary stone disease were excluded. Patients who underwent cholangioscopy using the first-generation Spyglass Legacy or those with no established final diagnosis were also excluded. Each participating center provided de-identified cholangioscopy procedural images and videos along with clinical information for eligible patients. To specify the final diagnosis, biliary strictures were labeled as malignant when there was a pathologically proven diagnosis obtained from cholangioscopy guided biopsy or brushing, endoscopic ultrasound (EUS)-guided biopsy, fine needle aspiration or a surgically resected specimen. Biliary strictures were labeled benign when no malignancy was observed after at least 12 months follow-up after cholangioscopy to avoid malignancy that was missed during the initial cholangioscopy. Patients without a final diagnosis of malignant biliary stricture and 12 month follow-up were excluded.

Data preparation and preprocessing: For each patient, we obtained demographic, clinical, and endoscopic data, in addition to images and video recordings from cholangioscopy. Demographic and clinical data included age, sex, risk factors, clinical symptoms, liver function tests, imaging findings, last follow-up visit, and death. Missing data for liver function tests were imputed using a non-parametric nearest neighbor algorithm. Endoscopic data included findings from any EUS-guided biopsy, information on visual impression during cholangioscopy, and findings from pathologic examination.

Two individuals annotated or reviewed each cholangioscopy image, including those extracted from videos, for key features suggestive of malignancy, including papillary mass, dilated and tortuous vessels, and ulceration. A senior investigator resolved discrepancies between the annotators. We processed the images to remove any patient information and other irrelevant markings such as company logos.

Image analysis: For each patient, we computed a summary feature to represent their images from cholangioscopy. We used a convolutional neural network (CNN) to compute the summary image feature. We trained the CNN to classify images for presence of the key features suggestive of malignancy. Across all images available from a patient, we computed the mean, kurtosis, and skew of predicted probabilities from the CNN as the summary image feature.

To classify images, we trained and evaluated three CNN architectures, including ResNet-18, Xception, and Inception-V3. ResNet-18 is a widely used CNN for image classification due to their simple architecture, high performance, and generalizability. The Inception-V3 network introduces different convolutional filters at the same level, which allows simultaneous extraction of features of low and high granularity. The Xception network extends Inception-V3 by incorporating depth-wise separable convolutions. Furthermore, Xception was evaluated in a previous study to analyze cholangioscopy images.

To train each CNN, we augmented the image data through random resizing, rescaling, rotation, cropping, blurring, and introducing Gaussian noise, color shifts, and brightness shifts. We evaluated learning rates of 0.001 and 0.0001, and batch sizes of 16 and 32, and used split-sample cross-validation to choose the best configuration for each network.

For cross-validation, we randomly assigned centers to train, validation, or test partitions such that 50% of patients in the sample were used for training, 20% for validation, and 30% for testing. Because we split the data by centers, patients in training or validation were not in the test set. Thus, our estimates of model performance evaluate generalizability or external validity. We used the data in the validation partition for model selection. We used the same data partitions when training the CNNs and developing clinical prediction models.

Clinical prediction models: To diagnose indeterminate biliary strictures as benign or malignant, we trained and evaluated three machine learning models using logistic regression (LR), random forests (RF), or gradient boosted machines (GBM) that are based on decision trees. As features to train the clinical prediction models, we used age, sex, weight loss, relevant past medical history such as primary sclerosing cholangitis, cystic biliary or liver disease, inflammatory bowel disease, parasitic infection, signs and symptoms including jaundice, weight loss and pain, and measures from liver function tests such as bilirubin, alanine transaminase (ALT), aspartate transaminase (AST), alkaline phosphatase (ALP). We predicted patients' final diagnosis of benign or malignant stricture. We trained models using clinical features, summary image features from ResNet-18, and combining clinical and summary image features from ResNet-18. In addition to data-driven feature selection for the clinical prediction model, we evaluated a pre-specified model that included age and jaundice.

For model selection using data in the validation partition, which included feature selection, we evaluated the L1 or L2 penalization for feature selection, reusing the previous fit to initialize the model, and regularization strength (varied 100 times between −5 and 5 on the log scale). For the RF and GBM, we varied the number of estimators (from 100 to 1000 in increments of 100), and number of minimum samples in each split and in each leaf (10, 20, or 30). Additionally, for the RF, we evaluated different stopping criteria (Gini index, entropy). For the GBM, we also varied the learning rate (from 1e-5 to 100 in steps of 10) and reusing the previous fit for initialization.

Statistical Analysis: We used t-tests and chi-squared tests for descriptive analyses. To evaluate model performance, we computed the estimates and 95% confidence intervals (CI) of sensitivity, specificity, area under the receiver operating characteristic curve (AUC), and positive and negative predictive values. To account for correlation because of multiple images from the same patients, we used multiple outputation to estimate measures of performance of the CNNs in the test set. The clinical prediction models used data at the patient-level, and thus, multiple outputation was not necessary. All statistical tests were two-sided, and we considered p-values less than 0.05 to be statistically significant. We used Python, and specifically PyTorch for CNNs and packages from Sci-kit Learn for the machine learning models.

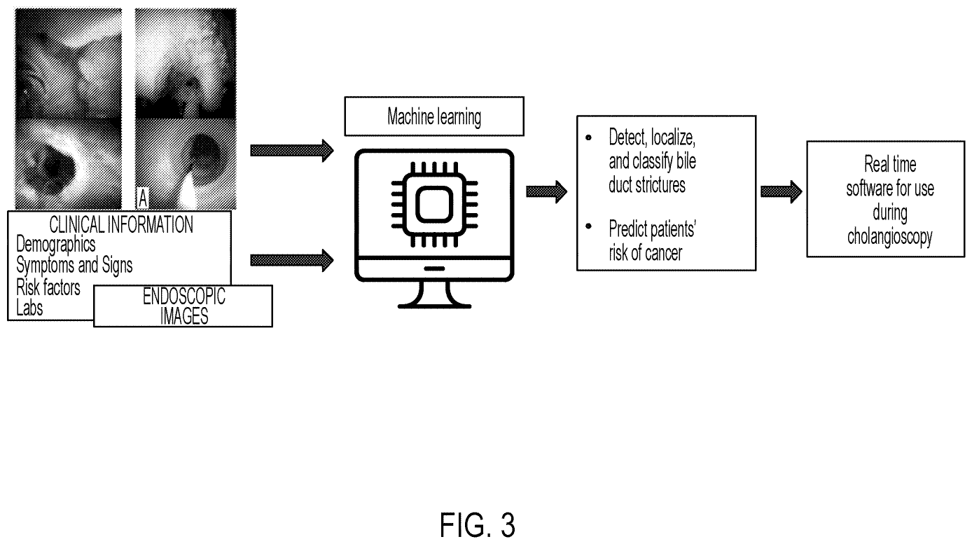

To further illustrate, FIG. 3 schematically depicts aspects of the machine learning process for classification of indeterminate biliary strictures using cholangioscopy images and a patient clinical data model described in this example.

### Results

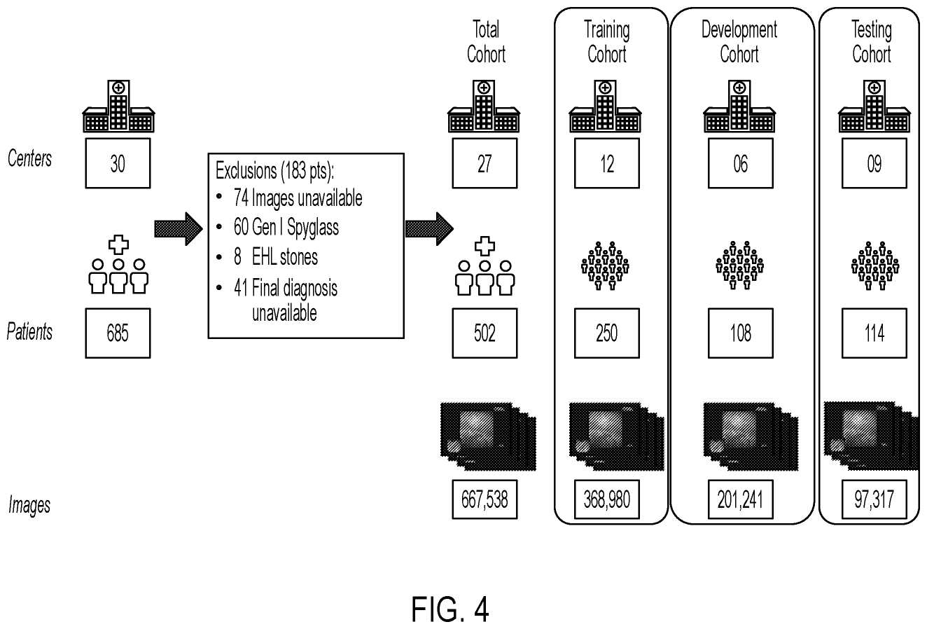

A total of 29 centers provided data on 685 patients who underwent cholangioscopy between **/20** and **/20**. We excluded 185 patients who did not meet eligibility criteria. We thus analyzed data from 502 patients from 27 centers (FIG. 4) of which 15 centers were in North America, seven in Asia, two in Europe, two Australia, and one in South America. Overall, 280 patients (55.8%) had a final diagnosis of malignant stricture, while 222 patients (44.2%) had a final diagnosis of a benign biliary stricture (Table 2.1). Most patients were males (n=277; 55%) with a mean age of 64.3±14.6 years. Patients with malignant strictures were more likely to be males (60% vs 49%), of older age (60.1 yr vs 67.6 yr), have jaundice (67% vs 33%), weight loss (45% vs 15%) and higher AST (107 vs 73) and ALT (147 vs 131) as compared to those with benign strictures (p<0.05).

TABLE 2.1
Baseline demographics and characteristics in the full cohort

| | Malignant | Benign | | |
|---|---|---|---|---|
| | Strictures | Strictures | Total | |
| | (N = 280) | (N = 222) | (N = 502) | p-value |
| Male; n (%) | 169 (60) | 108 (49) | 277 (55) | <0.05 |
| Age; mean ± sd | 67.6 ± 13.4 | 60.1 ± 15.0 | 64.3 ± 14.6 | <0.05 |
| Jaundice; n (%) | 188 (67) | 77 (35) | 265 (53) | <0.05 |
| Weight Loss; n (%) | 126 (45) | 33 (15) | 159 (32) | <0.05 |
| ALT; mean ± sd | 140.1 ± 147.6 | 98.4 ± 131.4 | 121.6 ± 142.1 | <0.05 |
| AST; mean ± sd | 107.2 ± 106.0 | 73.7 ± 82.2 | 92.4 ± 97.5 | <0.05 |
| ALP; mean ± sd | 434 ± 340.3 | 273.1 ± 213.8 | 362.8 ± 301.8 | <0.05 |

For split-sample cross-validation, we included 49.8% of patients from 44.4% of centers in the train partition, 21.5% of patients from 22.2% of centers in the validation partition, and 28.7% of patients from 33.3% of centers in the test partition. To train the CNNs, we analyzed a total of 667,358 images from cholangioscopy (368,980, 201,241, and 97,317 images from patients in the train, validation, and test partitions, respectively). There was no evidence of statistically significant differences between patients with and without a final diagnosis of malignancy, and among patients in the train, validation, and test data partitions.

Table 2.2 shows estimates of performance of the CNNs to classify images for presence of the key features suggestive of malignancy. All three CNNs had high AUC and sensitivity with moderate sensitivity. We chose ResNet-18 to provide the summary image feature included in the clinical prediction models.

TABLE 2.2
Performance characteristics of convolutional neural networks in detecting
biliary strictures based on cholangioscopy images. The estimate and
its standard deviation from multiple outputation are shown.

| Model | AUC | Sensitivity | Specificity | PPV | NPV |
|---|---|---|---|---|---|
| ResNet18 | 0.92 ± 0.000 | 0.90 ± 0.0003 | 0.78 ± 0.002 | 0.97 ± 0.000 | 0.50 ± 0.003 |
| Xception | 0.89 ± 0.000 | 0.92 ± 0.0002 | 0.63 ± 0.004 | 0.95 ± 0.0001 | 0.52 ± 0.004 |
| Inception_V3 | 0.92 ± 0.000 | 0.90 ± 0.0003 | 0.81 ± 0.001 | 0.97 ± 0.000 | 0.48 ± 0.003 |

Table 2.3 shows estimates of performance of models to predict a diagnosis in patients with indeterminate biliary strictures as benign or malignant. When combining clinical features and image features, the AUC in the test partition was 0.82 (95% CI, 0.71 to 0.94), 0.80 (95% CI, 0.67 to 0.93), and 0.80 (95% CI, 0.67 to 0.93) for LR, RF, and GBM, respectively. While all three models showed similar estimates of sensitivity, the specificity was 0.72 (95% CI, 0.49 to 0.88), 0.91 (95% CI, 0.62 to 0.98), and 0.67 (95% CI, 0.45 to 0.83) for LR, RF, and GBM, respectively. Combining image features and clinical features improved estimates of AUC over either set of features alone for all three machine learning models (Table 2.3). For the model including only age, jaundice and imaging features, the AUC, sensitivity, and specificity of LR were 0.88 (95% CI, 0.79 to 0.98), 0.88 (95% CI, 0.79 to 0.94), and 0.93 (95% CI, 0.70 to 0.99), respectively.

TABLE 2.3
Performance characteristics of convolutional neural network in
detecting malignant biliary strictures based on clinical and/or
image features Parentheses show 95% confidence intervals.

| | | | | | |
|---|---|---|---|---|---|
| Model | AUC | Sensitivity | Specificity | PPV | NPV |

Logistic regression

| | | | | | |
|---|---|---|---|---|---|
| Clinical | 0.75 | 0.81 | 0.56 | 0.68 | 0.71 |
| features | (0.67 to 0.84) | (0.71 to 0.88) | (0.44 to 0.67) | (0.58 to 0.77) | (0.58 to 0.82) |
| Image | 0.62 | 0.72 | 0.51 | 0.74 | 0.48 |
| features | (0.52 to 0.72) | (0.62 to 0.8) | (0.37 to 0.64) | (0.64 to 0.82) | (0.35 to 0.61) |
| Clinical + | 0.82 | 0.86 | 0.72 | 0.93 | 0.57 |
| image | (0.71 to 0.94) | (0.76 to 0.92) | (0.49 to 0.88) | (0.84 to 0.97) | (0.37 to 0.74) |
| features | | | | | |

Random forests

| | | | | | |
|---|---|---|---|---|---|
| Clinical | 0.72 | 0.76 | 0.58 | 0.77 | 0.56 |
| features | (0.64 to 0.81) | (0.66 to 0.83) | (0.44 to 0.71) | (0.68 to 0.85) | (0.42 to 0.68) |
| Image | 0.66 | 0.82 | 0.53 | 0.63 | 0.73 |
| features | (0.56 to 0.75) | (0.70 to 0.88) | (0.41 to 0.64) | (0.53 to 0.72) | (0.6 to 0.83) |
| Clinical + | 0.80 | 0.84 | 0.91 | 0.99 | 0.43 |
| image | (0.67 to 0.93) | (0.74 to 0.9) | (0.62 to 0.98) | (0.92 to 1.00) | (0.26 to 0.63) |
| features | | | | | |

Gradient boosted machine

| | | | | | |
|---|---|---|---|---|---|
| Clinical | 0.70 | 0.75 | 0.49 | 0.62 | 0.63 |
| features | (0.60 to 0.80) | (0.64 to 0.83) | (0.37 to 0.60) | (0.52 to 0.71) | (0.50 to 0.75) |
| Image | 0.59 | 0.66 | 0.40 | 0.62 | 0.44 |
| features | (0.5 to 0.69) | (0.56 to 0.75) | (0.28 to 0.53) | (0.52 to 0.71) | (0.32 to 0.58) |
| Clinical + | 0.80 | 0.87 | 0.67 | 0.90 | 0.61 |
| image | (0.67 to 0.93) | (0.77 to 0.93) | (0.45 to 0.83) | (0.80 to 0.95) | (0.41 to 0.78) |
| features | | | | | |

### Discussion

The use of AI including neural networks and machine learning methods has been rapidly growing in the various fields of medicine, including gastroenterology and luminal endoscopy. In hepatobiliary endoscopy and specifically, the use of AI in differentiating benign from malignant biliary strictures, the number of studies developing CNNs is on the rise. However, models are rarely evaluated in representative populations and settings not only for discrimination but also for calibration and clinical usefulness. Furthermore, most studies on cholangiocarcinoma used radiological imaging and endoscopic ultrasound images. Our findings show that images from cholangioscopy can be used to develop accurate clinical prediction models to diagnose biliary strictures. External validation of CNNs and three machine learning models showed they had an AUC of 0.80 or higher. The specificity of the image analysis and the clinical prediction models was lower than their sensitivity.

Our work addressed limitations in prior work on machine learning using cholangioscopy images to diagnose biliary strictures. In the other studies, the CNN was developed using data from 85 patients at one center. Although one study included multiple centers in the United States, the CNN was trained on data obtained from only a single center. Finally, in one study, model performance was over-estimated because the analysis did not consider correlation between multiple images from the same patient.

Our data were obtained from 30 centers across five continents. We partitioned data by centers such that patients whose data were used to develop the model were not used to test it. In other words, we evaluated external validity of the neural network and machine learning models in our study. Furthermore, we used multiple outputation to account for correlation in model performance on multiple images from patients. Finally, unlike other studies, we combined imaging features with clinical features and observed a resulting improvement in estimates of performance of the clinical prediction models.

In fact, our work lays the foundation for subsequent research for clinical translation of the CNN and prediction models. Future studies should be multicenter, which our work shows to be feasible, prospectively recruit patients with representative eligibility criteria, and evaluate calibration of model predictions in external samples and clinical usefulness of the models for decision-making. Analysis on clinical usefulness should include outcomes such as unwarranted surgery in misdiagnosed patients. Finally, the models should also be evaluated for their impact on clinical workflows.

Despite external validation using data from a large sample of patients in centers across the world, our study has limitations. Our data were retrospective because patient outcomes were recorded when data on the features were extracted from the medical charts. Thus, there was a risk of differential errors in data extraction. The cholangioscopy videos and images at all centers were not captured using the same protocol and systems. Thus, the quality of cholangioscopy videos and images was heterogeneous across the participating centers. Our findings show that the CNNs we trained are robust to the heterogeneous video and image quality. Data provided by the centers also differed in magnitude. While we split the data to ensure similar numbers of patients and images in all three partitions, the sensitivity of the CNN and the clinical prediction model to differences in representation of the participating centers is unclear. Our models showed low to moderate predictive values, which we hypothesize are a result of inaccuracy in annotations for a fraction of images provided to train the CNN originating from inherent inter-rater variability.

### Conclusion

In conclusion, our multicenter study aimed to develop and externally validate CNN algorithms to predict the nature indeterminate biliary strictures showed that incorporating clinical features to cholangioscopy images leads to a substantial improvement in clinical prediction models to diagnose malignant strictures.

Some further aspects are defined in the following clauses:

Clause 1: A computer-implemented method of classifying a disease state of a biliary stricture in a test subject, the method comprising: passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and, outputting from the electronic neural network a positive or negative disease state classification of the biliary stricture in the test subject indicated by the set of features extracted from the image data of the biliary stricture and the clinical data obtained from the test subject, thereby classifying the disease state of the biliary stricture in the test subject.

Clause 2: The computer-implemented method of Clause 1, wherein an accuracy level of the positive or the negative disease state classification output from the electronic neural network is higher than an accuracy level of the positive or the negative disease state classification output from another electronic neural network trained only on the plurality of sets of features extracted from image data of biliary strictures.

Clause 3: The computer-implemented method of Clause 1 or Clause 2, comprising generating a therapy recommendation for the test subject based upon the positive or negative disease state classification of the biliary stricture in the test subject output from the electronic neural network.

Clause 4: The computer-implemented method of any one of the preceding Clauses 1-3, comprising administering, or discontinuing administering, a therapy to the test subject based upon the positive or negative disease state classification of the biliary stricture in the test subject output from the electronic neural network.

Clause 5: The computer-implemented method of any one of the preceding Clauses 1-4, wherein the positive disease state comprises a cancerous disease state and wherein the negative disease state comprises a benign disease state.

Clause 6: The computer-implemented method of any one of the preceding Clauses 1-5, wherein the clinical data comprises demographic data, symptom data, physical examination data, or a combination thereof.

Clause 7: The computer-implemented method of any one of the preceding Clauses 1-6, wherein the clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level.

Clause 8: The computer-implemented method of any one of the preceding Clauses 1-7, wherein the image data comprises cholangioscopy images and/or videos of the biliary strictures obtained from the test subject and the reference subjects using a cholangioscope.

Clause 9: The computer-implemented method of any one of the preceding Clauses 1-8, wherein the biliary strictures of the reference subjects comprise indeterminate biliary structures.

Clause 10: The computer-implemented method of any one of the preceding Clauses 1-9, wherein the features extracted from the image data comprise indicators of papillary mass, dilated vessels, tortuous vessels, ulceration, or a combination thereof.

Clause 11: The computer-implemented method of any one of the preceding Clauses 1-10, wherein the features comprise numerical vectors.

Clause 12: The computer-implemented method of any one of the preceding Clauses 1-11, wherein the passing and outputting steps are performed substantially in real-time with obtaining the image data of the biliary stricture from the test subject.

Clause 13: A system for classifying a disease state of a biliary stricture in a test subject using an electronic neural network, the system comprising: a processor; and a memory communicatively coupled to the processor, the memory storing non-transitory computer-executable instructions which, when executed on the processor, perform operations comprising: passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and outputting from the electronic neural network a positive or negative disease state classification of the biliary stricture in the test subject indicated by the set of features extracted from the image data of the biliary stricture and the clinical data obtained from the test subject.

Clause 14: The system of Clause 13, wherein the instructions which, when executed on the processor, further perform operations comprising: generating a therapy recommendation for the test subject based upon the positive or negative disease state classification of the biliary stricture in the test subject output from the electronic neural network.

Clause 15: The system of Clause 13 or Clause 14, wherein an accuracy level of the positive or the negative disease state classification output from the electronic neural network is higher than an accuracy level of the positive or the negative disease state classification output from another electronic neural network trained only on the plurality of sets of features extracted from image data of biliary strictures.

Clause 16: The system of any one of the preceding Clauses 13-15, wherein the positive disease state comprises a cancerous disease state and wherein the negative disease state comprises a benign disease state.

Clause 17: The system of any one of the preceding Clauses 13-16, wherein the clinical data comprises demographic data, symptom data, physical examination data, or a combination thereof.

Clause 18: The system of any one of the preceding Clauses 13-17, wherein the clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level.

Clause 19: The system of any one of the preceding Clauses 13-18, wherein the image data comprises cholangioscopy images and/or videos of the biliary strictures obtained from the test subject and the reference subjects using a cholangioscope.

Clause 20: The system of any one of the preceding Clauses 13-19, wherein the biliary strictures of the reference subjects comprise indeterminate biliary structures.

Clause 21: The system of any one of the preceding Clauses 13-20, wherein the features extracted from the image data comprise indicators of papillary mass, dilated vessels, tortuous vessels, ulceration, or a combination thereof.

Clause 22: The system of any one of the preceding Clauses 13-21, wherein the features comprise numerical vectors.

Clause 23: A computer readable media comprising non-transitory computer-executable instructions which, when executed by at least one electronic processor, perform at least: passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and, outputting from the electronic neural network a positive or negative disease state classification of the biliary stricture in the test subject indicated by the set of features extracted from the image data of the biliary stricture and the clinical data obtained from the test subject.

Clause 24: The computer readable media of Clause 23, wherein the instructions which, when executed by the electronic processor, further perform operations comprising: generating a therapy recommendation for the test subject based upon the positive or negative disease state classification of the biliary stricture in the test subject output from the electronic neural network.

Clause 25: The computer readable media of Clause 23 or Clause 24, wherein the positive disease state comprises a cancerous disease state and wherein the negative disease state comprises a benign disease state.

Clause 26: The computer readable media of any one of the preceding Clauses 23-25, wherein the clinical data comprises demographic data, symptom data, physical examination data, or a combination thereof.

Clause 27: The computer readable media of any one of the preceding Clauses 23-26, wherein the clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level.

Clause 28: The computer readable media of any one of the preceding Clauses 23-27, wherein the image data comprises cholangioscopy images and/or videos of the biliary strictures obtained from the test subject and the reference subjects using a cholangioscope.

Clause 29: The computer readable media of any one of the preceding Clauses 23-28, wherein the biliary strictures of the reference subjects comprise indeterminate biliary structures.

Clause 30: The computer readable media of any one of the preceding Clauses 23-29, wherein the features extracted from the image data comprise indicators of papillary mass, dilated vessels, tortuous vessels, ulceration, or a combination thereof.

Clause 31: The computer readable media of any one of the preceding Clauses 23-30, wherein the features comprise numerical vectors.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A computer-implemented method of classifying a disease state of a biliary stricture in a test subject, the method comprising:
passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and,
outputting from the electronic neural network a positive or negative disease state classification of the biliary stricture in the test subject indicated by the set of features extracted from the image data of the biliary stricture and the clinical data obtained from the test subject, thereby classifying the disease state of the biliary stricture in the test subject.

2. The computer-implemented method of claim 1, wherein an accuracy level of the positive or the negative disease state classification output from the electronic neural network is higher than an accuracy level of the positive or the negative disease state classification output from another electronic neural network trained only on the plurality of sets of features extracted from image data of biliary strictures.

3. The computer-implemented method of claim 1, comprising generating a therapy recommendation for the test subject based upon the positive or negative disease state classification of the biliary stricture in the test subject output from the electronic neural network.

4. The computer-implemented method of claim 1, comprising administering, or discontinuing administering, a therapy to the test subject based upon the positive or negative disease state classification of the biliary stricture in the test subject output from the electronic neural network.

5. The computer-implemented method of claim 1, wherein the positive disease state comprises a cancerous disease state and wherein the negative disease state comprises a benign disease state.

6. The computer-implemented method of claim 1, wherein the clinical data comprises demographic data, symptom data, physical examination data, or a combination thereof.

7. The computer-implemented method of claim 1, wherein the clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level.

8. The computer-implemented method of claim 1, wherein the image data comprises cholangioscopy images and/or videos of the biliary strictures obtained from the test subject and the reference subjects using a cholangioscope.

9. The computer-implemented method of claim 1, wherein the biliary strictures of the reference subjects comprise indeterminate biliary structures.

10. The computer-implemented method of claim 1, wherein the features extracted from the image data comprise indicators of papillary mass, dilated vessels, tortuous vessels, ulceration, or a combination thereof.

11. (canceled)

12. The computer-implemented method of claim 1, wherein the passing and outputting steps are performed substantially in real-time with obtaining the image data of the biliary stricture from the test subject.

13. A system for classifying a disease state of a biliary stricture in a test subject using an electronic neural network, the system comprising:
a processor; and
a memory communicatively coupled to the processor, the memory storing non-transitory computer-executable instructions which, when executed on the processor, perform operations comprising:
passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and
outputting from the electronic neural network a positive or negative disease state classification of the biliary stricture in the test subject indicated by the set of features extracted from the image data of the biliary stricture and the clinical data obtained from the test subject.

14. The system of claim 13, wherein the instructions which, when executed on the processor, further perform operations comprising:
generating a therapy recommendation for the test subject based upon the positive or negative disease state classification of the biliary stricture in the test subject output from the electronic neural network.

15. The system of claim 13, wherein an accuracy level of the positive or the negative disease state classification output from the electronic neural network is higher than an accuracy level of the positive or the negative disease state classification output from another electronic neural network trained only on the plurality of sets of features extracted from image data of biliary strictures.

16. The system of claim 13, wherein the positive disease state comprises a cancerous disease state and wherein the negative disease state comprises a benign disease state.

17. (canceled)

18. The system of claim 13, wherein the clinical data from the test and reference subjects comprises sex, age, presence or absence of jaundice, presence or absence of weight loss, presence or absence of pain, medical history of primary sclerosing cholangitis, cystic biliary or liver disease, inflammatory bowel disease, and/or parasitic infection, a bilirubin level, an alanine transaminase (ALT) level, an aspartate transaminase (AST) level, and/or an alkaline phosphatase (ALP) level.

19. The system of claim 13, wherein the image data comprises cholangioscopy images and/or videos of the biliary strictures obtained from the test subject and the reference subjects using a cholangioscope.

20. The system of claim 13, wherein the biliary strictures of the reference subjects comprise indeterminate biliary structures.

21. The system of claim 13, wherein the features extracted from the image data comprise indicators of papillary mass, dilated vessels, tortuous vessels, ulceration, or a combination thereof.

22. (canceled)

23. A computer readable media comprising non-transitory computer-executable instructions which, when executed by at least one electronic processor, perform at least:
passing a set of features extracted from image data of the biliary stricture and clinical data obtained from the test subject through an electronic neural network, wherein the electronic neural network has been trained on a set of training data that comprises a plurality of sets of features extracted from image data of biliary strictures and clinical data obtained from reference subjects, wherein the image data of the biliary strictures and the clinical data obtained from the reference subjects are each labeled with a positive or negative disease state ground truth classification for a given reference subject, and wherein one or more predictions for a positive or negative disease state classification for the given reference subject are made based on the image data of the biliary strictures and the clinical data obtained from the given reference subject, which predictions are compared to the ground truth classification for the given reference subject when the electronic neural network is trained; and,
outputting from the electronic neural network a positive or negative disease state classification of the biliary stricture in the test subject indicated by the set of features extracted from the image data of the biliary stricture and the clinical data obtained from the test subject.

24.-31. (canceled)
